(19) United States
(12) Patent Application Publication
(10) Pub. No.: US 2006/0041262 A1
(43) Pub. Date: Feb. 23, 2006

(54) Interlaced wire for implants

(76) Inventors: Jay Wynn Calvert, Newport Beach, CA (US); Steven Michael Greenthal, Buena Park, CA (US)

Correspondence Address: Pepper Hamilton LLP, One Mellon Center, 50th Floor, 500 Grant Street, Pittsburgh, PA, 15219, US

(21) Appl. No.: 11/133,400

(22) Filed: May 19, 2005

Related U.S. Application Data
(60) Provisional application No. 60/572,331, filed on May 19, 2004.

(51) Int. Cl. A61B 17/58

(52) U.S. Cl. 606/76

(57) ABSTRACT

The disclosure is directed to a material for in vivo implantation. The material includes a compressed interlaced wire shaped into a form having a porous structure suitable for tissue ingrowth, the interlaced wire compressed from an original starting density to a final density, the final density being greater than the original starting density.

### RELATED APPLICATION AND CLAIM OF PRIORITY

This application claims priority to, and incorporates herein by reference, U.S. Provisional Patent Application No. 60/572,331, entitled “Compressed Knitted Metallic Wire Shapes for Use in Bone Repair and Replacement,” and filed May 19, 2004.

### BACKGROUND OF THE INVENTION

1. Field of the Invention

The present invention is generally directed to devices useful for reconstructive surgery. In particular, the present invention is directed to a non-sintered interlaced wire usable in implants, wherein it is desirable that the wire is shaped into a form having a porous structure suitable for tissue ingrowth.

2. Description of Related Art

Artificial joints and bones, used to repair, replace, or reconstruct defective bones are known in orthopedic surgery. Devices suitable for such replacement must contain several properties. Devices must be biocompatible, provide structure strength to bear loads produced in a skeleton structure, be flexible enough to transfer load and strain to existing bone supporting the artificial device, and must provide areas in which connective bone tissue may attach to the device. Existing development of such prosthetic devices have generally consisted of providing a solid structure resembling a bone made of metal or biocompatible plastics such as polyethylene and providing a second component attached to the solid structure, such as a porous metal material for the site of tissue ingrowth.

For example, U.S. Pat. No. 3,992,725 to Homsey discloses an implantable material having a preferred composition of carbon and graphite fibers, and optionally metallic and ceramic fibers, bonded together by sintered polytetrafluoroethylene (PTFE). These materials are bonded to the surface of an implant to stabilize the implant by fostering ingrowth of bony tissue.

U.S. Pat. No. 4,064,567 to Burstein et al. discloses a mesh sheath woven of metal wire, such as titanium, stainless steel and chrome cobalt. The sheath is used as a reinforcing element with bone cement to arrest crack formation and maintain the shape and integrity of the cement.

In U.S. Pat. No. 3,906,550 to Rostoker et al., a porous fiber metal structure is produced by molding and sintering short metal fibers which are preferably kinked prior to being cut into short fibers. In practice, the short metal fibers are mechanically molded into the desired precise shapes using constraining dies and moving punches to produce a three-dimensionally mechanically interlocked network of fibers which is then sintered.

A porous metal surface layer is coated by a plasma spraying process on a dense base of the same metal to form a prosthetic device according to the teachings in U.S. Pat. No. 3,605,123 to Hahn. The density of the porous surface layer is greatest (substantially pore-free) at the interface with the base and becomes progressively larger towards the exterior surface.

A stocking-like prosthetic device is also disclosed in German Offenlegungschrift No. 2842847 to Adolph Voorhoeve. According to this device, a funnel-shaped mesh prosthesis can be formed from intersecting and interconnecting steel wires or filaments which permits some deformation in a direction perpendicular to the surface of the mesh and to a lesser degree in the plane of the mesh.

A porous compound material for prosthetic devices is also disclosed in UK Patent Application GB No. 2 059 267A. The compound material includes at least one layer of metal wire mesh joined to a metal substrate by means of metallurgical bonds at the points of contact.

A metal wire mesh sheet used in endoprosthetics is disclosed in U.S. Pat. No. 4,693,721 to Ducheyne. Ducheyne describes titanium or other biocompatible metal fibers having a diameter of from about 20 to about 200 μm, a length of from about 2 to 50 mm and a length-to-diameter ratio of at least about 100 formed into a flexible and deformable mass, for example, a sheet, of interlocked fibers, which may be sintered for additional coherence, to a thickness of from about 0.5 to 30 mm.

Another disclosure of a filling member for bone cavities is U.S. Pat. No. 5,665,119 to Koller. Koller describes a tubular member made from wire, preferably titanium wire, which is manufactured on a round knitting machine, which is inserted into the cavity of a pressing tool having a pressing mold and die. The tubular member is plastically deformed by the die introduced into the cavity, so that the member assumes the shape predetermined by the pressing mold, and thus, is transformed to produce a tubular member which can be inserted as a filling member into bone cavities, for example, as a medullary space barrier.

Another metal wire structure is disclosed in U.S. Pat. No. 5,397,359 to Mittelmeier et al. Mittelmeier et al. discloses a metal wire structure for endoprosthetics formed of a sintered hollow mesh knitting of elastic metal wires, the knitting being preferably manufactured in the manner of a “rete milanese”. The metal wire structure is either used as a coating of an endoprosthesis or, after having been sintered together, as a bone replacement piece. Such a metal wire structure allows to obtain a good anchorage in the bone, since the pore size of the meshes may be adapted to the local conditions, on one hand, and on the other hand, the remaining elasticity of the meshes allows a suitable transition from said prosthesis to the bone.

Therefore, it remains a goal in the art to design and implement an artificial device that provides enough structural strength to bear loads that are produced, provides enough bending compliance to effectively transfer loads and to simulate existing skeletal flexibility and to provide a surface on which connective bone or tissue cells may attach and grow.

Additionally, the present invention relates to the field of bone repair and replacement, including a bone plate and screw system. It is current practice in orthopedic surgery to use plating systems for joining portions of a broken bone, or for fusion of portions of separate bones. Such systems are composed essentially of plates and screws for aligning and holding the bone portions in a desired position relative to one another. Plating systems have usefulness in the spine, and have general skeletal use on the flat bones, such as the scapula and the pelvis by way of example, and for use on tubular bones, such as the humerus, ulna, radius, femur, and tibia by way of example.

Plating systems may also be used in the splinting of a bone fracture with a plurality of bone screws. Several recent designs have incorporated bioabsorbable materials such as plastics into the plate and screw assembly. For example, U.S. Pat. No. 6,540,746 to Buhler et al. describes the use of an elastic cushion to separate the bone plate and bone screws. Additionally, the Boehringer Company, Ingelheim, Germany, manufactures plastics such as polylactides, a bioabsorbable plastic used in bone plate and screw fixations.

Problems associated with existing plating systems have included hardware breakage, hardware loosening, inability to gain adequate fixation, and distraction pseudoarthrosis where the plate will not allow the bone portions to come together over time resulting in a failure to get solid bone healing. These occurrences may cause problems, be associated with surgical failure, and require further surgical procedures to repair the damage, remove the failed hardware, and/or to reattempt skeletal stabilization.

Plates are usually provided to the surgeon for use in sets having a range of sizes so as to provide for such features as biological variability in size, the numbers of segments to be joined, and the length of the portions of bone to be joined. By way of example, it would be common for a plating system for use on the anterior cervical spine and for joining from two to five vertebrae to comprise of forty to sixty plates.

Based on a consideration of the features of the known plating systems, there remains a need for an improved plating system comprised of a material having the following combination of features: enough structural strength to bear loads that are produced, enough bending compliance to effectively transfer loads and to simulate existing skeletal flexibility and to provide a surface on which connective bone or tissue cells may attach and grow.

### SUMMARY OF THE INVENTION

The present invention discloses an interlaced wire product for use in reconstructive surgery, such as bone repair or replacement surgical implantation and procedures which exhibits strength, flexibility, and that provides a surface structure which promotes tissue ingrowth.

The device or interlaced wire products described in the present application may be suitable in a number of surgical applications. As such, suitable applications of the interlaced wire products include but are not limited to bone repair, replacement, bone fusions, bone cavity incisions, prosthetic devices, implant devices, plate and screw devices, and artificial joint construction such as hip and shoulder joints. The term “reconstructive surgery” as used herein will thus refer to this broad category of surgical applications, including orthopedic surgery, spine surgery, cranial surgery, pelvic surgery, chest wall reconstruction, trunk reconstruction, abdominal reconstruction, hand surgery and podiatric surgery, unless so specifically limited otherwise.

In an embodiment, the present invention is directed to a device useful for reconstructive surgery. The device includes a non-sintered interlaced wire shaped into a form suitable for implantation into a mammal. The form includes a portion of porous structure suitable for tissue ingrowth.

The non-sintered interlaced wire may be compressed into the form of the device. The interlaced wire may not be sintered as part of the compression. The non-sintered interlaced wire may be a mesh sheet. The mesh sheet may be fibrous. The device may be flexible. The non-sintered interlaced wire may include a plurality of wires, each wire having a diameter of about 0.001 inches to about 0.03 inches. The non-sintered interlaced wire may be selected from the group including cobalt-chromium alloy, cobalt, molybdenum, gold alloy, titanium, titanium alloy, tantalum, tantalum alloy, stainless steel, niobium, niobium alloy, or a combination thereof. The non-sintered interlaced wire may include a first component and a second component, wherein the first component and second component may be interengaged to form the device.

Another embodiment of the present invention is directed to a non-sintered plurality of interlaced fibers shaped into a biocompatible device. The plurality of interlaced fibers retain their structural integrity and form a porous structure suitable for tissue ingrowth. The non-sintered plurality of interlaced fibers may be compressed into the shape of the biocompatible device. The plurality of interlaced fibers may preferably be of the same length. Each fiber of the plurality of interlaced fibers may have a diameter of about 0.001 inches to about 0.03 inches, preferably about 0.001 inches to about 0.02 inches.

A further embodiment includes a method of forming a device for reconstructive surgery. The method includes interlacing a plurality of wires to form an interlaced wire and compressing the interlaced wire to form a shape. The compressed interlaced wire forms a porous structure suitable for tissue ingrowth.

The method may include a step of machining the compressed interlaced wire. The method may include knitting the plurality of wires in a circular knitting machine or the like. The method may further include compressing the interlaced wire, each wire of the plurality of wires having a diameter of about 0.001 inches to about 0.03 inches to a crushed density of about 10% to about 50%. The method may further include a step of providing a coating on at least one of the plurality of wires. The interlaced wire may be selected from the group including cobalt-chromium alloy, cobalt, molybdenum, gold alloy, titanium, titanium alloy, tantalum, tantalum alloy, stainless steel, niobium, niobium alloy, or a combination thereof. Preferably, the interlaced wire may be a titanium alloy, stainless steel alloy or a combination thereof. Each wire of the plurality of wires may have a diameter of about 0.001 inches to about 0.03 inches.

Another embodiment includes a biocompatible, deformable material used in reconstructive surgery. The material may be compressed to a shape having the form of a porous structure suitable for tissue ingrowth. The material having been compressed from an original starting density to a final density, the final density may be greater than the original starting density.

The material may be metallic, polymeric or any other suitable material. The material may be compressed into a disk, cylindrical, ball-ended, hemispheric, conical, truncated conical, cubical, or free-form shape. The material may be non-sintered. The material may have a diameter of about 0.001 to about 0.15 inches. The material may be used in reconstruction applications such as implant, reconstruction plate, and load bearing applications. The material may be selected from the group including cobalt-chromium alloy, cobalt, molybdenum, gold alloy, titanium, titanium alloy, tantalum, tantalum alloy, stainless steel, niobium, and niobium alloy or a combination thereof. The resulting structure may have a pore size of about 100 to about 500 microns, preferably about 175 to about 500 microns and more preferably, about 200 microns to about 300 microns.

Another embodiment of the invention includes a biocompatible, deformable interlaced wire used in reconstructive applications. The interlaced wire includes a material compressed from an original starting density to a final density, the final density being greater than the original starting density to form a shape having a porous structure. The interlaced wire may be compressed into a disk, cylindrical, ball-ended, hemispheric, conical, truncated conical, cubical, or free-form shape. The interlaced wire may form a mesh sheet. The interlaced wire may have a diameter of about 0.001 inches to about 0.03 inches. The interlaced wire may be used in reconstruction applications such as implant, reconstruction plate, and load bearing applications. The material of the interlaced wire may be selected from the group including of cobalt-chromium alloy, cobalt molybdenum, gold alloy, titanium, titanium alloy, tantalum, tantalum alloy, stainless steel, niobium, niobium alloy or any combination thereof. The interlaced wire having a diameter of about 0.001 to about 0.03 inches may be compressed to a crushed density of about 10% to about 50%. The porous structure may include a pore size of about 175 microns to about 500 microns, preferably about 175 microns to about 500 microns, and more preferably, about 200 microns to about 300 microns.

A further embodiment includes a material for in vivo implantation. The material includes an interlaced wire shaped. The interlaced wire may be shaped into a form having a porous structure suitable for tissue ingrowth by compressing the interlaced wire from an original starting density to a final density, the final density being greater than the original starting density. The interlaced wire may be compressed into a disk, cylindrical, ball-ended, hemispheric, conical, truncated conical, cubical, or free-form shape. The interlaced wire may have a diameter of about 0.001 inches to about 0.03 inches. The interlaced wire may be used in reconstruction applications including implant, reconstruction plate, load bearing and mesh sheet applications. The interlaced wire may be selected from the group including cobalt-chromium alloy, cobalt, molybdenum, gold alloy, titanium, titanium alloy, tantalum, tantalum alloy, stainless steel, niobium, niobium alloy, or a combination thereof. The interlaced wire may be non-sintered and having a diameter of about 0.001 inches to about 0.03 inches may be compressed to a crushed density of about 10% to about 50%. The structure may include a pore size of about 175 microns to about 500 microns, preferably about 200 microns to about 500 microns, and more preferably, about 200 microns to about 300 microns.

Yet another embodiment is directed to a biocompatible wire for use in reconstructive surgery. The wire may be compressed to a shape having a porous structure suitable for tissue ingrowth from an original starting density to a final density, the final density being greater than the original starting density.

Another embodiment of the present invention includes a biocompatible bone implant device including one or more compressed interlaced wire shapes. Another embodiment of the present invention may be an interlaced wire product for use in bone repair or replacement surgical implantation and procedures which exhibits strength, flexibility, and that provides a surface structure which promotes tissue ingrowth including one or more compressed interlaced wire shapes.

Aspects and applications of the present invention will become apparent to the skilled artisan upon consideration of the detailed description of the invention and the figures accompanying the description, which follow.

### DESCRIPTION OF THE DRAWINGS

The invention will now be described in greater detail with reference to specific embodiments thereof and with the aid of the accompanying drawings in which:

FIG. 1 illustrates a device according to an embodiment of the present invention;

FIG. 2 illustrates a device according to another embodiment of the present invention;

FIG. 3 illustrates a device according to a further embodiment of the present invention; and

FIG. 4 illustrates a device according to another embodiment of the present invention.

### DETAILED DESCRIPTION

For simplicity and illustrative purposes, the principles of the invention are described by referring mainly to an embodiment thereof. In the following description, numerous specific details are set forth in order to provide a thorough understanding of the invention. It will be apparent however, to one of ordinary skill in the art, that the invention may be practiced without limitation to these specific details. In other instances, well known methods and structures have not been described in detail so as not to unnecessarily obscure the invention.

It must also be noted that as used herein and in the appended claims, the singular forms “a”, “an”, and “the” include plural reference unless the context clearly dictates otherwise. It is noted that the term “interlaced wire” encompasses a plurality of fibers or a single strand fiber and the interlaced wire may be coiled, looped, knitted, woven, patterned and the like. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

In the following description various embodiments of an interlaced wire, along with methods of construction and use are provided. The examples hereinbelow refer to interlaced wire usable in reconstructive surgery, for example, implants, including bone repair and replacement applications. However, it is to be understood that the invention, both methods and articles are not limited to such end applications.

The present invention is generally directed to a device useful in reconstructive surgery. The device may include implants, reconstruction plates, load-bearing devices, mesh sheets and the like. In particular, the invention is directed to an interlaced wire shaped into a form suitable for implantation into a mammal.

The interlaced wire may include a plurality of fibers or wires. The plurality of fibers preferably may be of substantially the same length. Each fiber of the interlaced wire may have a diameter of about 0.001 inches to about 0.03 inches, preferably about 0.001 inches to about 0.020 inches. The diameter of the interlaced wire may vary depending on the desired application and/or the desired properties. The interlaced wire may be of any configuration. For example, the interlaced wire may have a repetitive configuration such as a spring-like configuration, or alternatively may have a random configuration. Alternatively, the interlaced wire may have a predetermined pattern. The interlaced wire of the present invention does not require thermal processing, for example, sintering to form the porous structure of the device.

The interlaced wire may be of any material. In particular, U.S. Pat. No. 3,992,725, herein incorporated by reference in its entirety, describes the properties of a material used in surgical implantations. For example, the material may include properties such as bio-compatibility, resistance to chemical migration when implanted, stability for autoclaving, porosity for tissue ingrowth, and resiliency. The material utilized in the present invention may include cobalt-chromium alloy, cobalt, molybdenum, gold alloy, titanium, titanium alloy, tantalum, tantalum alloy, stainless steel, niobium, niobium alloy or any combination thereof. Preferably, the interlaced wire may include titanium alloys, stainless steel alloys or a combination thereof. Titanium may be preferred because of its biocompatibility as well as its low modulus of elasticity which increases the overall flexibility of a component produced from the titanium fibers. Alternatively, the interlaced wire may be of another material such as polymers, composites or the like that have biocompatible properties. In an embodiment, the interlaced wire may include a coating on the outer surface of the interlaced wire or each fiber of the interlaced wire. The interlaced wire may also be plated, tempered or otherwise treated to facilitate manufacturing.

The interlaced wire may be shaped into a form suitable for implantation in a mammal. The interlaced wire shaped form may be resilient. Various interlaced wire shape forms of this invention may be further deformed to a desired interlaced wire shape. Resulting interlaced wire components of the present application may also exhibit appropriate flexibility once implanted into the skeletal system.

The form includes a porous structure suitable for tissue ingrowth. The porous structure preferably may be flexible and/or deformable. The porous structure includes pores varying in size from about 175 microns to about 500 microns, preferably about 200 microns to about 500 microns and more preferably, about 200 microns to about 300 microns. In an alternate embodiment, the form may include a portion of the porous structure.

The interlaced wire may be compressed into the form of an implant as described hereinbelow in further detail. By compressing the interlaced wire, the interlaced wire achieves a higher density product with increased strength while maintaining the interlaced wire's original structural integrity. The higher density product includes a random orientation of the interlaced wire to form the porous structure. In an alternate embodiment, the interlaced wire may be compressed to a pre-determined pattern forming the porous structure. The interlaced wire may be compressed to a crushed density of about 10% to about 50%. The term “crushed density” as used herein describes the density of the device or final formed shape in comparison to a solid of the same shape. In other words, the crushed density value represents the percentage of the amount of the compressed interlaced wire to an amount of a solid form of the same shape.

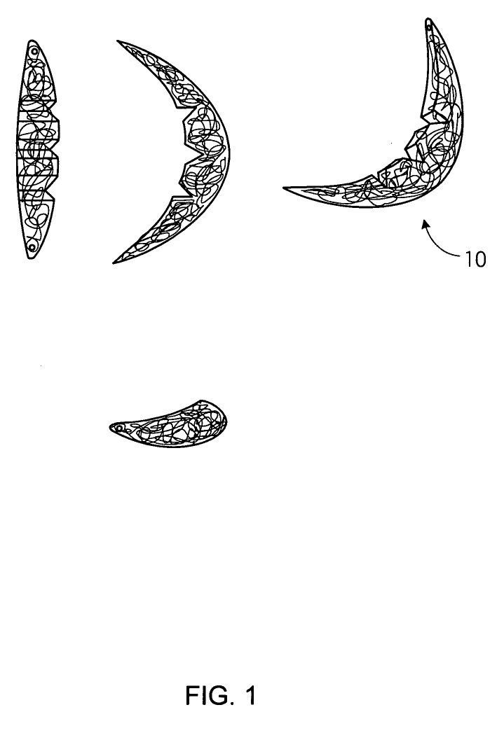

FIG. 1 illustrates a device in accordance with one embodiment of the present invention. Specifically, FIG. 1 illustrates components of interlaced wire formed in various shapes for a chin implant 10. The chin implant does not bear a significant amount of load or strain and is thus not under excessive stresses. The shape formed provides a fibrous network or porous structure that is suitable for tissue ingrowth.

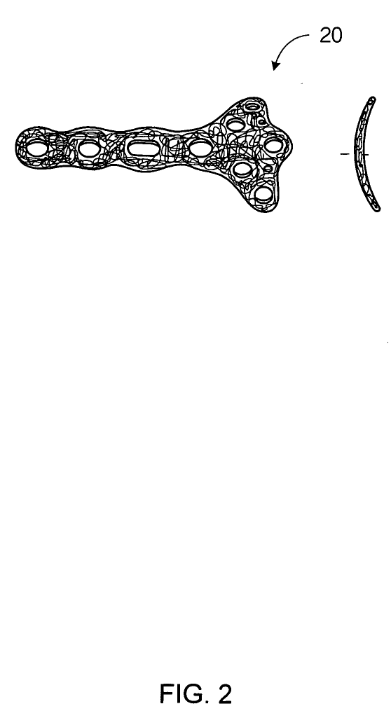

FIG. 2 illustrates an example of a device according to another embodiment of the invention. FIG. 2 illustrates a reconstruction plate 20. For example, the present tooling methods and compressed products may be used in a number of applications, including a bone plate and screw fixation system. The use of bone plate and bone screw fixation systems for treating injuries to bones is well established. In most instances, a bone plate may be engaged to a bone with the plate over and surrounding the bone injury area. The bone plate may preferably be affixed to the bone by bone screws or other similar fasteners inserted through holes in the bone plate and into the bone itself. The screws may be tightened after penetrating the bone so that the bone plate holds the bone to be treated in place in order to insure proper healing.

Early fixation devices tended to be applicable only to long bone injuries with some limited uses for lower lumbar spinal injuries and disorders. The use of plate/screw fixation systems expanded, however, to include uses for more spinal injuries and fusion of vertebrae including fixation devices for treating cervical vertebrae injuries. Some plate and screw systems encounter a variety of problems which lead to less than optimal results. These problems include, amongst others, “backout.” Backout is the exhibited tendency of bone screws, which affix the bone plate to the bone(s), to loosen with respect to both the plate and bone resulting in poor fixation, fusion and ultimately, healing. Essentially, this loosening of the bone screw causes the screw to work itself out of the bone into which it is implanted. This results in the bone plate being poorly fixed in place thus becoming devoid of its fixation capabilities. Usually, backout is caused by the stress of bodily movement. While such loosening can be benign if limited in scope, it more often leads to complications such as complete failure of the fixation device or incomplete bone fusion. Backout is particularly prevalent in areas of high bodily stress, such as the spine.

To alleviate backout and its associated problems, current systems employ the device processed according to an embodiment of the present invention. The compressed interlaced wire may have a concave surface or other suitable shape. The plate may conform to the shape of a bone on which it is placed in vivo. The component may act as the plate in a typical plate and screw fixation system. The compressed metal component may contain one or more screws affixed through the shape and into the bone. The screws may be inserted into the compressed metal component by means described above such that the screws become self-locking. A benefit of using the compressed interlaced wire of the present invention as the plate material is that the resulting plate and screw assembly provides flexibility in the skeleton system. Additionally, the plate and screw system provides a suitable site for tissue regrowth upon implantation. A plate and screw system of the present invention may include the use of any suitable screw machined with the compressed metal wire component.

The use of a shaped compressed interlaced wire metal wire component in connection with bone repair or replacement treatments and procedures, may be suitable in any part of the body including, hands, feet, elbow, face, knee, shoulders, hip, skull, or spine. Specifically, plate and screw assemblies have been used throughout the skeletal system to effectuate repair. Additionally, the compressed device or shaped interlaced wire may simulate any skeletal component or portion thereof.

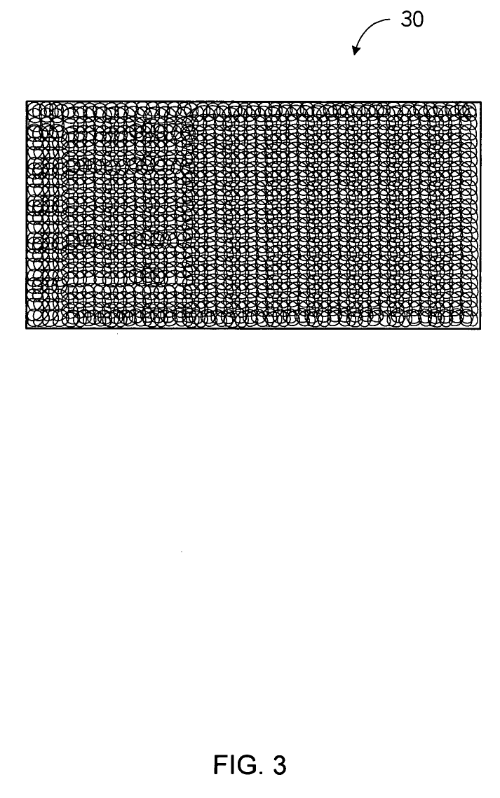

FIG. 3 illustrates a device according to another embodiment of the present invention. FIG. 3 illustrates a mesh sheet 30. The mesh sheet 30 may include biologically inert fibers that are interwoven forming a porous structure suitable for tissue growth. In an embodiment, the biologically inert fibers may be metallic fibers. Alternatively, the biologically inert fibers may be wire that forms a fibrous network. In an alternate embodiment, the mesh sheet may be compressed.

A mesh sheet may be formed by knitting a wire in a circular knitting machine or the like with a relatively high knitting tension to form a tubular shell or flat interlaced wire fabric (or sheet) having an original dimension. The knitted or interlaced fabric may then be compressed into a shape that is smaller than the original dimension of the tubular or flat interlaced fabric and exhibits greater density. The device may further be machined into a final product.

The mesh sheet compressed shape according to one embodiment of the present invention has a broad range of applications in reconstructive surgery. For example, the mesh sheet may be used in plate and screw fixation devices, bone repair and replacement devices, including load bearing applications, bone reconstruction applications, revision surgery of the hip joint and other joint prostheses as well as implant procedures, and generally for any procedure where a normal regrowth of bone tissue cannot be expected.

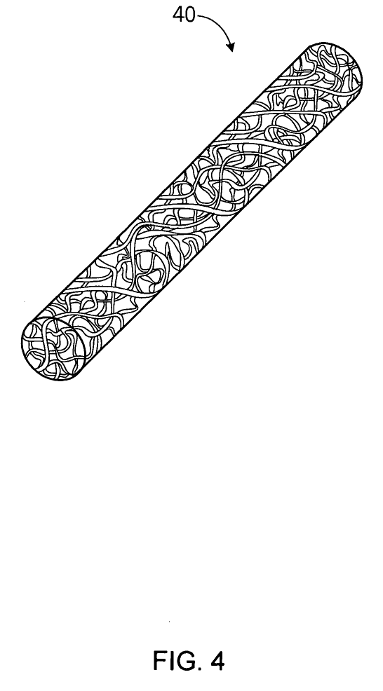

FIG. 4 illustrates a device in accordance with a further embodiment of the present invention. FIG. 4 illustrates a device 40 used in a load bearing device. Specifically, FIG. 4 illustrates an interlaced wire compressed to a rod shape having a porous structure. The rod may then be utilized to replace, for example, a femoral shaft. Due to the load bearing application and the need to be able to withstand heavier loads and more acute or intense stresses, an interlaced wire having a large diameter may be utilized. In an embodiment, the interlaced wire may include a single strand wire that is coiled or looped upon itself. Any diameter of the interlaced wire may be used, preferably a larger diameter wire, for example, about 0.15 inch may be utilized to provide increased strength properties in the device. Preferably, the interlaced wire has a diameter of about 0.06 to about 0.25 inches and more preferably, about 0.06 inches to about 0.15 inches.

A load bearing orthopedic prosthesis including a stem having a distal end adapted be inserted into an existing joint socket or an artificial joint socket. For example, the distal end may be substantially spherical in shape and may be formed by compressing an interlaced wire. The load bearing orthopedic prosthesis may then be placed into another artificial implant component or may be fitted into an existing socket joint according to known implant procedures. Alternatively, an interlaced wire compressed component may be used as the stem component in an artificial orthopedic device.

Another example of a device in accordance with the present application includes hip prosthesis applications. For example, when severe hip joint problems are encountered, it may be necessary to replace the hip joint, either the ball or the socket or both. The large upper leg bone, or femur has a long lower main portion, with a head or ball connected by a neck portion angled inward toward the hip socket from the upper end of the main portion of the femur. One generally used hip joint replacement technique involves removal of the head and neck of the femur, and the insertion of a long angled and tapered metal prosthesis into the central intramedulary canal at the open upper end of the main straight portion of the femur. This femoral prosthesis typically includes a relatively small metal ball at its upper end which mated with a small plastic socket mounted on the hip side of the joint.

On the socket side of the joint, referred to as the “acetabular”, prostheses may be employed which use a plastic cup to mate with the femoral component. It has been determined that these plastic acetabular components are subject to considerable wear. Additionally, known hip replacement materials include metal-to-metal joints.

In a typical artificial hip joint, there may be a femoral component, an acetabular component with a head or ball-like end to simulate the ball of the ball and socket hip joint. Typically, the femoral component may be a metal or plastic replacement prosthesis and the acetabular component is a metal replacement prosthesis. Devices of shaped interlaced wires according to the present invention may be suitable as the femur, the head of the femur, the socket side, acetabular members, and other bone members according to several embodiments of the present invention.

Another embodiment of the invention includes a method of forming a device for reconstructive surgery. The method includes interlacing a plurality of wires and compressing the interlaced wire to form a shape. Additionally, further processing of the interlaced wire may occur.

A step of the method includes interlacing a plurality of wires. This may be done, for example, in a circular knitting machine with a relatively high knitting tension to form a tubular or flat knitted metal wire fabric having an original dimension. Any machine as understood by one skilled in the art may be used. Generally, the interlacing step may result in a flat sheet of meshed wire or a sheet that is slightly rolled into a tubular form. Circular knitting machines which produce a knitted tube of wire fabric are known in the art, and have been used for many years to process wire filament.

The knitting of wire mesh presents several processing variables. In such knitting, a relatively high knitting tension may be required to form the wire into the desired fabric. This compounds the difficulty presented by the relative lack of stretchability of wire, which makes it prone to breakage if snags develop during knitting. Knitting variables which may be adjusted according to end applications include wire diameter, knitting tension, and knitting speed.

A knitted wire sheet may be pre-formed into any shape and subject to any further processing steps according to an embodiment of the invention, or conversely utilized by several embodiments of the present invention in its pre-formed shape.

The next step includes compressing the interlaced wire to form a shape of a device having a porous structure. In compressing the knitted metal wire fabric into a shape, the compressed shape may be smaller than the original dimension of the tubular or flat interlaced wire fabric and exhibits a greater density than the original wire fabric. The original starting material may include a single piece of interlaced wire. Alternatively, the starting material may include a number of interlaced wires. The structural integrity of the interlaced wire is maintained during the compression step. Additionally, the form of the porous flexible structure is achieved through mechanical processing of the interlaced wire and does not include any thermal processing for forming the implant, such as thermal processing, including sintering.

Compression pressure may be of any suitable range. The density of the resulting compressed metal shape depends on the compression pressure and the thickness of the interlaced wire. The variables of the compression pressure, shape density, and wire diameter may all be adjusted according to the final end application of the compressed metal shape.

After the metal fibers are knitted, it may be directly laid down or into on a suitable surface, e.g., a mold. The amount of pressure to be applied perpendicular toward the wire in the mold depends on various factors such as the type of wire, thickness of the wire and porosity of the sheet, but generally pressures in the range of from about 4 MPa to about 200 MPa may be used.

A primary advantage of manufacturing product from compressed wire mesh is the ability of the product to flex. This flexibility is controlled by varying the wire strength, hardness, diameter and compression density.

Compressed interlaced wire shapes of the present invention or the final products they are incorporated into before insertion into the skeletal structure may include properties that simulate bone elasticity and strength properties. Such orthopedic properties are generally known. See Reilly, D. T. and Burstein, A. H., “The elastic and ultimate properties of compact bone tissue”, J. Biomechanics , 8, 393-405, 1975. For example, a human femur exhibits the following properties: Longitudinal strength, 205 MPa; strain 0.019; Compressive transverse strength, 131 MPa; strain 0.028-0.087; Tensile longitudinal strength, 135 MPa, strain 0.031; Tensile transverse strength, 53 MPa, strain 0.007.; and Shear strength, 65-71 MPa.

An example of a compression step may include a cylindrical mold cavity loaded with a specific amount, by weight, of knitted wire mesh produced in a circular knitting machine as described above. The wire may then be compressed using a movable pushing tool to press the wire into a stationary cavity to a specific total thickness. This procedure preferably allows control of final density of finished product. The product after compression with the push tool may be disk-shaped or cylinder-shaped depending on finished thickness.

Another embodiment of the compression step, may include a mold and push configured with concave contact surfaces that produce a compressed product with convex shape. Varying the amount of wire mesh, as well as the compression pressure determines the final part thickness and density. A push configured with a convex contact surface may produce a compressed product with a concave shape. In this manner a compressed metal wire shape with a concave surface suitable to be placed on top of a cylindrical bone may be produced. Other skeletal surfaces may be simulated as well. An example of a finished part produced by this tooling may be a hemispheric-shape or a ball-ended cylinder shape depending on finished thickness, which may be varied according to pressure applied. Optionally, a fully round, ball-shaped compressed wire form made in the described manner, such as with the compression tool described may be used as an insert.

Yet another embodiment of the compression step of the present invention, includes tooling that may be used to create a freeform product. The tooling may include a base or bottom plate with a cavity end shape cut in to the plate. Above the base plate may be a cavity plate that includes a profile of the finished part cut through the plate. The top component may be the pusher tool that fits in the profile cut in the cavity plate and has the cavity end detail cut into it. In use, the cavity plate is attached to the base plate. The wire mesh in this tooling may be compressed with the pusher, with the finished part having the bottom plate shape on the product bottom and the pusher end shape on the product top after compressing. The finished product may then be removed from the tooling after compressing.

The mold cavity, guide block and push may be formulated to any suitable shape. As such, shapes that resemble bones and skeleton parts or their surfaces may be created in one embodiment of the invention. A specific compressed metal wire shape may resemble a femur shape, including a main stem portion and a head portion in one continuous free form compressed shape. Such compressed metal wire shapes may be used in artificial bone implant devices.

Another compressed shape suitable for use as an implant device according to another embodiment of the invention is two or more compressed metal wire shapes. For a combination product example, one compressed metal wire shape may be produced with the one of the tooling described above and the other may be an insert made separately as described above. This round insert in the center of the product may be made to a different design criteria, such as larger diameter wire compacted to a low density to act as a hollow core, within a complete crushed wire product of smaller wire diameter and greater density. Inserts may be manufactured with completely different specifications, materials and procedures to create finished products that perform different functions at different depths or areas, based on the requirements, such as final specification of bone implant devices in vivo.

For example a wire metal compressed shape may be compressed into a cylindrical shape having a specific density and relatively small diameter. This cylindrical shape may then simulate the bone marrow portion of an artificially constructed bone. As such, another compressed metal wire component may be formed around the cylindrical shape with different density and diameter to simulate an artificial bone. Additionally, the compressed cylindrical shape may be used as an inclusion into another prosthetic device of another material, such as plastic, before implanting into the skeletal system of an orthopedic patient.

For example, in another embodiment, a fully round, ball-shaped compressed wire form is used in a bone implant device, in which the ball-shaped compressed wire is used as the head of the femur in a replacement hip. As known in the art, the hip is a ball-and-socket joint where the head of the femur articulates with the cuplike acetabulum of the pelvic bone. As such, the ball-shaped compressed wire form may be combined with a femur-like article for inclusion into a prosthetic device. In another embodiment of the present invention, a compressed ball-shaped wire mesh product may be used in an artificial shoulder assembly.

The two main bones of the shoulder are the humerus and the scapula (shoulder blade). The joint cavity is cushioned by articular cartilage covering the head of the humerus and face of the glenoid. The scapula extends up and around the shoulder joint at the rear to form a roof called the acromion, and around the shoulder joint at the front to form the coracoid process. The end of the scapula, called the glenoid, meets the head of the humerus to form a glenohumeral cavity that acts as a flexible ball-and-socket joint. The joint is stabilized by a ring of fibrous cartilage surrounding the glenoid called the labrum.

A compressed ball-shaped wire mesh product may simulate the head of the humerus in an artificial shoulder assembly. Another compressed wire mesh shape may simulate the glenohumeral cavity in an artificial shoulder assembly. The compressed ball-shaped wire mesh product may include an encapsulated tube protruding therefrom which simulates humerus. The artificial humerus may be comprised of any suitable material and manufactured in suitably known manners.

A suitable product comprised of the compressed shapes may also consist of many different layers and inserts to satisfy many specific requirements in a finished prosthetic device. Inserts may be made of compacted knitted wire or solid materials such as rods, tubes, screws, nails, and flat bars as well as special shapes may be included within a product.

Optionally, a product may contain more than one compressed interlaced wire shapes. Different density materials may be used in different areas for different mechanical requirements. As explained above, the insert inclusion may simulate the hip as a ball-and-socket joint where the head of the femur articulates with the cuplike acetabulum of the pelvic bone.

In another embodiment, a product includes a pre-manufactured tube encapsulated within, first a low-density, large-diameter wire core and second, a high-density small-diameter wire outer compressed shell. This pre-manufactured tube encapsulated within a compressed ball-shaped wire mesh product may be a phalanges-simulating prosthetic device. The tube may be any suitable artificial phalange, metacarpal, metatarsal, or other skeleton component. Additionally this pre-manufactured tube may be a receiving tube for a screw or another suitable attachment device. Other suitable articles to be included in a compressed knitted metallic shape include screws, nails, needles, hinges, washers, nuts and other surgical articles. By encapsulating a compressed knitted metallic shape around a hollow tube, the resulting density of the shape may be varied.

Products according to other embodiments of the invention may also be manufactured with holes and void areas by use of cores or inserts placed within the compression chamber prior to compression and subsequently removed from the product after compression.

Another embodiment of the compression step includes a machining step, for example, utilizing a tool to make a product with integral holes. A designated portion of knitted metallic wire may be pushed onto a tooling platform with pins affixed perpendicularly outward thereto. As the knitted metallic wire is compressed onto the pins of the tooling, a compressed shape with integral holes is formed. The tapered core pins cause the wire mesh to separate and conform to the pin body during wire mesh compression.

After compressing, additional steps may include one or more of machining or processing steps. Examples of such processing may be a lamination, a combination of one compressed shape with additional compressed wire components, a combination of the compressed metal shape with solid objects, insertion of integral holes into compressed metal shapes, or combinations of several of the machining steps. By processing different layers or inserts of compressed metal wire shapes into a final product, countless configurations of compressed metal shapes may be assembled. It is understood that other elements made by the method of the invention in different configurations would be compressed in a similar manner.

For example, a compressed knitted metallic shape may be formulated into a spherical shape around an encapsulated tube. The shape may then act as an insert to another spherical shape which is formed around both the insert and encapsulated tube. The hole may be machined to intersect a tube that was encapsulated during the compression step. This hole may be machined into the part with a drill, or any other suitable tooling. A tool may then be forced into the compressed wire shape to produce one or more holes.

Another example includes self-locking screw holes machined after compression process completion. By nature of the random wire locations, which result when the metal wire is compressed into a shape, machining a hole through the compressed product would produce a hole with random exposed wire ends. The hole may subsequently be distorted out of natural shape during screw insertion, causing the random wires to encroach upon the diameter of the screw, and subsequently causing the screw to resist reverse rotation, thus locking the screw.

In this regard methods of the present invention may be employed to produce a flexible plate and screw fixation device that results in a locking screw assembly in which the plate material may be an interlaced wire component being compressed to provide a suitable surface for the regrowth of tissue. The compressed metal wire shape may be formulated into a suitable “plate” which may include a concave surface to be placed on top of bones. One or more bone screws may be used in connection with the “plate” as provided. The one or more screws penetrates existing skeletal components upon insertion into a patient.

The bone repair and replacement treatments and method of the present invention may be used in connection with any known procedure and/or materials. For example, suitable materials typically used in orthopedic surgery may be used in connection with compressed metal components to effectuate bone repair or replacement.

For example, suitable biological materials may be used including bone replacement materials, such as biocompatible polymers; autograft, allograft, demineralized bone matrix (DBM) products; bone growth factors, such as transforming growth factor-beta (TGF-β), fibroblast growth factors (FGF), allogeneic-derived growth factors, blood-extracted growth factors, and recombinant platelet-derived growth factors (RHPDGF); tissue replacement materials, such as chondrocyte transplants, collagen scaffolds, meniscal allografts, ligamentous autografts, ligamentous allografts, tendinous allografts, biologic scaffolds and patches, and growth factors and gene therapies; and synovial fluids, such as hyaluronic acid and other synovial fluid replacement products.

Synthetic biomaterials may also be used in various embodiments of the present invention. These include fixation materials, such as bioresorbable polymers, structural calcium phosphates and sulfates, composite materials, calcium phosphate cements; and replacement materials, such as synthetic bone fillers, bioactive implant coatings, bioresorbable polymer scaffolds.

In addition to the above stated bone replacement and repair applications, compressed wire mesh techniques may also be used to manufacture small hardware such as screws, washers, nuts and various shapes, according to embodiments of the present invention. Very small wire diameter and high compression rates would be required to form small parts. The resultant parts are strong and flexible and exhibit consistent porosity structures.

The above-described flexible metallic knitted wire mesh shapes, according to several embodiments of the present invention may thus be used in bone repair or replacement surgical operations and procedures. The shapes exhibit variable strength, flexibility, and provide a porous surface structure which promotes tissue ingrowth.

### EXAMPLE 1

Example 1 is a static structural device, specifically a chin implant. A chin implant is designed to create the shape of a chin but not necessarily bear any significant load. The implant incorporates with the bone of the mandible and the overlying soft tissue. In order for this to occur, the implant is composed of an interlaced wire having a diameter of about 0.014 inches and compressed to about 30% crushed density to the shape of the chin implant. Porosity of the porous structure formed is approximately 80% with a mean pore size of about 250 microns. The implant may be perfused with cells if it was desired to have more bone growth within the body of the implant.

### EXAMPLE 2

Example 2 is a flexible mesh sheet of compressed wire that may be utilized, for example, as a replacement for a polypropylene mesh, in the abdomen, or as a flexible titanium mesh on the skull. Wire that is either knitted or woven, or a single strand wire is crushed into a flat sheet that is approximately 0.07 inches thick. The formed structure of the mesh sheet is about 50% porous and the pore size is about 225 microns to about 275 microns. The mesh sheet is made of interlaced wire having about 0.0035 inch thickness at about 50% crushed density.

### EXAMPLE 3

Example 3 is directed to a load bearing device including bone replacements such as would be used to replace a section of the femoral shaft. The load bearing application has a higher tensile strength to accommodate the weight of the patient's body and the stresses common to the femoral shaft. This implant is made of coiled or looped wire having a diameter of about 0.06 inches at about 15% crushed density.

### EXAMPLE 4

In example 4, the crushed wire is crushed to the shape of an osteosynthesis fracture healing plate. These plates will have a degree of flexibility that allows the plate to function to fix fractures and still have a degree of flexibility that allows the plate to not be too rigid. This demonstrates a significant difference from traditional rigid fixation as this plate has added flexibility. The interlaced wire for this device has a thickness of about 0.008 inches at about 40% crushed density.

All of the embodiments disclosed and claimed herein can be made and executed without undue experimentation in light of the present disclosure. While the devices and methods of this invention have been described in terms of preferred embodiments, it will be apparent to those of skill in the art that variations may be applied to the composition, methods, and in the steps or in the sequence of steps of the methods described herein, without departing from the concept, spirit and scope of the invention. More specifically, it will be apparent that certain further tooling and manufacturing steps may be performed on the metal wire mesh products of the present invention. All such tooling and manufacturing steps apparent to those skilled in the art are deemed to be within the spirit, scope and concept of the invention as defined by the appended claims.

What is claimed is:

1. A device useful for reconstructive surgery comprised of a non-sintered interlaced wire shaped into a form suitable for implantation into a mammal, said form having a portion of porous structure suitable for tissue ingrowth.

2. The device of claim 1, wherein the non-sintered interlaced wire is compressed into the form of the device.

3. The device of claim 2, wherein the non-sintered interlaced wire having a diameter of about 0.001 inches to about 0.03 inches is compressed to a crushed density of about 10% to about 50%.

4. The device of claim 1, wherein the non-sintered interlaced wire comprises a plurality of wires, each wire having a diameter of about 0.001 inches to about 0.03 inches.

5. The device according to claim 1, wherein the non-sintered interlaced wire is selected from the group comprising cobalt-chromium alloy, cobalt, molybdenum, gold alloy, titanium, titanium alloy, tantalum, tantalum alloy, stainless steel, niobium, niobium alloy, or a combination thereof.

6. The device according to claim 1, wherein the non-sintered interlaced wire is a titanium alloy, stainless steel alloy, or a combination thereof.

7. A method of forming a device for reconstructive surgery, comprising:
interlacing a plurality of wires to form an interlaced wire; and
compressing the interlaced wire to form a shape.

8. The method according to claim 7, wherein the compressed interlaced wire forms a porous structure suitable for tissue ingrowth.

9. The method according to claim 7, further comprising the step of machining the compressed interlaced wire.

10. The method according to claim 7, wherein the interlaced wire is selected from the group comprising cobalt-chromium alloy, cobalt, molybdenum, gold alloy, titanium, titanium alloy, tantalum, tantalum alloy, stainless steel, niobium, niobium alloy, or a combination thereof.

11. The method according to claim 7, further comprising the step of providing a coating on at least one of the plurality of wires.

12. The method according to claim 7, wherein each wire of the plurality of wires has a diameter of about 0.001 inches to about 0.03 inches.

13. The method according to claim 7, further comprising compressing the interlaced wire, each wire of the plurality of wires having a diameter of about 0.001 inches to about 0.03 inches to a crushed density of about 10% to about 50%

14. A material for in vivo implantation, the material comprising an interlaced wire shaped into a form having a porous structure suitable for tissue ingrowth by compressing the interlaced wire from an original starting density to a final density, the final density being greater than the original starting density.

15. The material according to claim 14, wherein the material is non-sintered.

16. The material according to claim 14, wherein the wire is selected from the group comprising of cobalt-chromium alloy, cobalt, molybdenum, gold alloy, titanium, titanium alloy, tantalum, tantalum alloy, stainless steel, niobium, niobium alloy or a combination thereof.

17. The material according to claim 14, wherein the wire has a diameter of about 0.001 inches to about 0.03 inches.

18. The material according to claim 14, wherein the wire is compressed to a crush density of about 10% to about 50%.

19. The material according to claim 14, wherein the interlaced wire is used in reconstruction applications selected from the group consisting of implant, reconstruction plate, load bearing applications and mesh sheet applications.

20. The material according to claim 14, wherein the structure has a pore size of about 175 microns to about 500 microns.
